(19) United States
(12) Patent Application Publication
(10) Pub. No.: US 2002/0156392 A1
(43) Pub. Date: Oct. 24, 2002

(54) Method and apparatus for inspecting biological rhythms

(75) Inventors: Kuniharu Arai, Pittsburgh, PA (US); Mitsuru Yoneyama, Kanagawa (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Mitsubishi Chemical Corporation, Tokyo (JP)

(21) Appl. No.: 09/799,027

(22) Filed: Mar. 6, 2001

(51) Int. Cl. A61B 5/04

(52) U.S. Cl. 600/546

(57) ABSTRACT

An inspection apparatus for determining a neurological disease at an earlier stage of disease with high probability. The inspection apparatus includes a biological rhythm detector unit for non-invasively measuring biological rhythm information from the acceleration involved in a repetitive rhythmic movement by using a detector attached on a body of a subject, an information collector unit for recording and/or storing the resulting biological rhythm information, and an analyzer for analyzing variation and aging in the physiological function of the subject.

### BACKGROUND OF THE INVENTION

(a) Field of the Invention

The present invention relates to a method and an apparatus for inspecting biological rhythms. More particularly, the present invention relates to a biological rhythm inspection method and apparatus for judging a variation or aging in physiological function of a subject at an earlier stage of disease.

(b) Description of the Prior Art

Recently, with increasing complexity and variety as well as graying of the society, the number of people who lose the rhythm of their body and mind due to stress or allergy and are suffered from a neurological disease such as dementia grows with each passing year. This has become a serious issue of public concern. Such a variation or aging in physiological function has been conventionally diagnosed in accordance with the expert knowledge or experience of doctors. In these cases, clinical observations have been employed through diagnosis, consultation, intelligence tests, and motility tests, which are provided by doctors. Image observations in accordance with image information on the brain using the MRI have been also employed.

However, these clinical and image observations cannot be applied until the variation or aging in physiological function has become considerably apparent. Recent years have not yet seen a basic remedy for many neurological diseases, typified by Alzheimer's disease. The development of such means as for detecting the diseases at an earlier stage with a high probability would provide an advantage of making it possible to take precautionary measures against or prevent further development of the diseases at an earlier stage. This would save the patient and the patient's family from disastrous situations.

On the other hand, as can be seen in the recent walking or fitness boom, such an idea has been established among people that they should improve their physical strength or activate their brain cells by making use of exercises readily available to them in daily life.

In this context, a variation and aging in physiological function may be diagnosed and determined at an earlier stage objectively in a quantitative manner only by monitoring an exercise such as gait which is familiar to a person. This would be very convenient and significantly advantageous. In the past years, such an apparatus as is called a life monitor device has been reported which is to measure the heartbeat, posture, and rhythm of finger tapping of a person. However, most parts of these methods require some restraints to make the measurements reliable. The restraints include disposing the electrodes or sensors in close contact with the skin or fixing them to a particular portion of the body, or having to conduct the inspection in a place capable of satisfying certain conditions such as an inspection room of a hospital.

### SUMMARY OF THE INVENTION

In view of the above problems, it is an object of the present invention to provide a biological rhythm inspection apparatus and method which allow a variation or aging in physiological function to be determined at an earlier stage with a high probability

The present invention provides, in one aspect thereof, a biological rhythm inspection apparatus including: a biological rhythm detector for non-invasively measuring a muscle activity in a repetitive rhythmic movement caused by a voluntary movement involving a body movement of a subject; an information collector unit for recording or storing biological rhythm information obtained from the biological rhythm detector; and an analyzing unit for analyzing a variation or aging in physiological function based on the biological rhythm information.

The present invention also provides, in another aspect thereof, a biological rhythm inspection method including the steps of: measuring a repetitive rhythmic muscle activity caused by a voluntary movement involving a body movement of a subject, by using a biological non-invasive rhythm detector attached on a body of the subject, to obtain biological rhythm information; comparing the biological rhythm information with reference data; and analyzing a variation or aging in physiological function of the subject based on the result of the comparison.

In accordance with the present invention, the variation or aging in physiological function of the subject can be analyzed at an earlier stage of the disease by comparing the current biological rhythm information with a reference data.

The term “body movement” as used herein means a movement including a shift or travel of the subject with respect to the environment thereof, which may include a simulated shift or travel of the subject. The reference dada may be past data obtained from the subject as time-series data, or data obtained from other persons such as an able-bodied person.

For example, the reference data collected from the able-bodied categorized by sex and age can be compared with the current data of the subject, thereby making it possible to determine the level of the physiological function of the subject caused by a variation or aging in physiological function of the subject. Alternatively, the data of the subject can be accumulated as time-series data, thereby allowing the current level of the subject to be judged with respect to the past data, and additionally, allowing the future level to be predicted

In the present invention, it is to be understood that a variation or aging in physiological function of a subject results from, for example, ages or diseases as well as any causes resulting from the frequency of physical conditions and fatigue caused in daily life. The inspection apparatus and method of the present invention can be preferably applied to, among other things, the determination or prediction of neurological diseases such as Alzheimer's disease, cerebrovascular dementia (e.g., multiple cerebral infarction), and Parkinson's disease.

The above and other objects, features and advantages of the present invention will be more apparent from the following description, referring to the accompanying drawings.

### BRIEF DESCRIPTION OF THE DRAWINGS

FIG. 1 is a block diagram illustrating the configuration of a biological rhythm inspection apparatus;

FIG. 2 is a block diagram illustrating the configuration of an information collecting apparatus;

FIG. 3 is a schematic view illustrating an exemplary apparatus for measuring gait rhythms;

FIGS. 4(a) to 4(f) show graphs illustrating the results of a fractal analysis;

FIGS. 5(a) to 5(f) show graphs showing the results of a scaling analysis based on principal component analysis;

FIGS. 6(a) and 6(b) each show the analytical results of able-bodied persons and patients with a reduced neurological physiological function;

FIGS. 7(a) and 7(b) are graphs each showing an autocorrelation function of the acceleration data on the biological rhythm of able-bodied persons and patients with a reduced neurological physiological function;

FIGS. 8(a) and 8(b) are graphs each showing a chaotic attractor of the acceleration data on the biological rhythm of able-bodied persons and patients with a reduced neurological physiological function; and

FIGS. 9(a) and 9(b) are graphs each showing the result of a fractal analysis and a differential function thereof, respectively.

### DETAILED DESCRIPTION OF THE PREFERRED EMBODIMENTS

Now, the present invention will be described below in more detail with reference to the accompanying drawings.

Referring to FIG. 1, a biological rhythm inspection apparatus 10 according to an embodiment of the present invention includes a biological rhythm detector unit 12 for non-invasively measuring, as biological rhythm information, phenomena involved in repetitive rhythmic movements. For example, the phenomena include a change in physical strength, a spatial change in position of the body, a sound given off from the body, a change in wave energy such as electromagnetic waves or in subtle energy, or a change in field around the body. The apparatus also includes an information collector unit 14 for recording and storing the biological rhythm information obtained from the biological rhythm detector unit 12. The apparatus further includes an information processing unit 16 for analyzing the biological rhythm information recorded in the information collector unit 14 to output data for determining a variation or aging in physiological function at an earlier stage. The apparatus also includes an output unit 18 for outputting the analyzed results provided by the information processing unit 16.

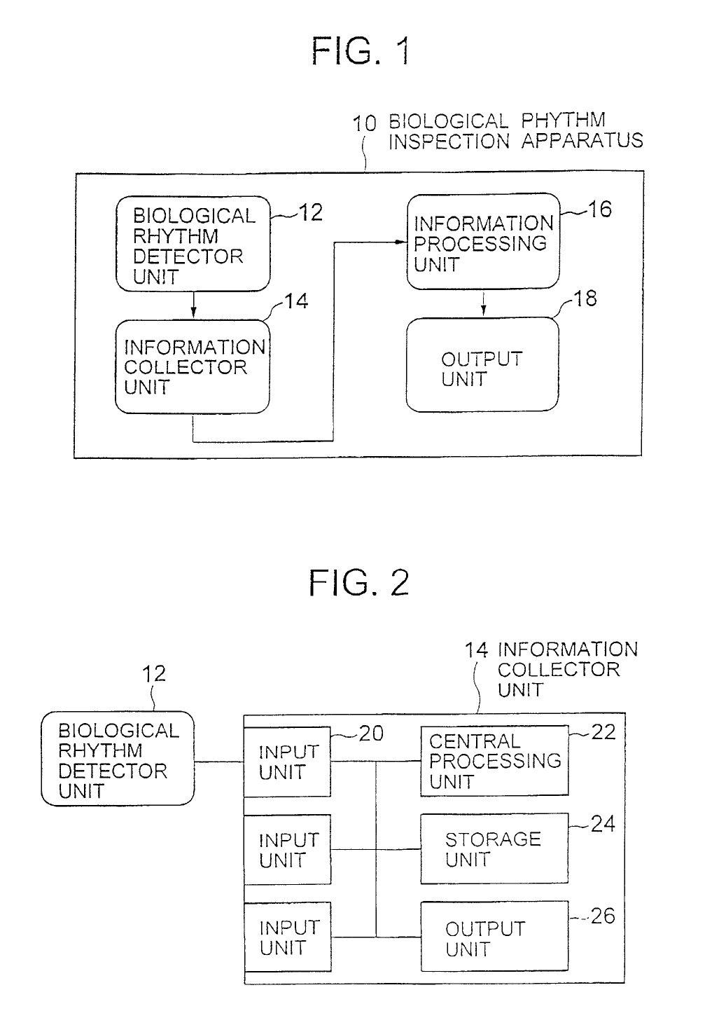

Referring to FIG. 2, the information collector unit 14 includes an input unit 20, a central processing unit 22 connected to the input unit 20, a storage unit 24, and an output unit 26.

The information processing unit 16 analyzes the biological rhythm information recorded in the information collector unit, for example, acceleration data on a muscle activity. In this case, the analysis is conducted in accordance with the fractal analysis, FFT analysis, autocorrelation function analysis, nonlinear analysis (chaotic attractor), Lyapunov exponent, or scaling analysis based on principal component analysis. In this analysis, included may be the step of comparing the biological rhythm information with the past data of the subject or the data of other persons which is stored in advance or input additionally from outside in the information processing unit 16.

The biological rhythm detector unit 12 and the information collector unit 14 may be integrated with each other. Alternatively, a signal detected by the biological rhythm detector unit 12 may be successively sent to the information collector unit 14 by radio or the like.

The information collector unit 14 and the information processing unit 16 may be integrated with each other. Alternatively, the information collector unit 14, after the biological rhythm information has been recorded thereon, may be connected to the information processing unit 16. In this case, the connection includes dismounting a removable recording medium such as a memory card from the information collector unit 14 and mounting the medium to the information processing unit 16. Also included is the transmission of the information to the information processing unit 16 by radio or over a cable such as a telephone line or optical fiber

In the present embodiment, the repetitive rhythmic movement caused by voluntary movement involving a person's travel means a body exercise which the subject can consciously control to some extent and yet about which the subject is not necessarily conscious at all times during the exercise. Preferred examples include a rhythmic exercise involving shift or travel such as gait, running, swimming, or bicycling (cycling). The main reasons for this include as follows:

(1) The data analysis methods of the present invention preferably use data collected for a time length greater than a specified time length, such as collected during a repetitive rhythmic exercise repeated 100 times or more to determine a variation or aging in physiological function with high reliability. The aforementioned exercises are available in daily life without making special efforts and can be continued for a long time to some extent, thus matching to the aforementioned object.

(2) It is preferable to measure and analyze a rhythmic exercise frequently and continually to determine a variation or aging in physiological function. The aforementioned exercises are available in daily life and practiced periodically as a way of improving physical strength, thus matching to the aforementioned object.

(3) Since the exercises preferably require involvement of the whole body, the biological rhythm detector unit can be attached to anywhere on the body, thereby allowing a more non-invasive measurement than the measurement such as of finger tapping which requires the movement of only fingers.

(4) The exercises are controlled by CPG (Central Pattern Generator) which is present in a neurological circuit including the spinal cord, reflecting the neurological health status as well as requiring the whole body. Therefore, the exercises also reflect sensitively the condition of a wide range of body and mind such as stress, fatigue, or a localized pain on the body.

The present invention measures a repetitive rhythmic movement due to a voluntary movement involving the subject's movement or travel. The travel of the subject means a subject's displacement relative to the surrounding environment. More specifically, the travel includes ordinary exercises such as gait, running, swimming, bicycling (cycling) in which the subject's body is moved. In addition to this, the travel includes exercises using a gait-simulating device such as a treadmill in which the gait surface is moved, a running-simulating device similar to the gait device, a bicycling-simulating device, or a swimming pool with flowing water.

The biological rhythm detector 12 is a sensor for non-invasively measuring a repetitive rhythmic movement due to a voluntary movement of the subject. The detector employs, for example, a small acceleration sensor as a meter. The small acceleration sensor is attached to a portion capable of detecting the repetitive activity of muscle due to a voluntary movement to measure the acceleration involved in the repetitive rhythmic movement.

An acceleration signal is converted to a voltage signal, which is in turn received in the storage unit 24 through an A/D converter (not shown) provided in the input unit 20 of the information collector unit 14 at a sampling frequency which is sufficient to reproduce the original rhythm.

No restrictions are imposed on the place of attachment and connection of the biological rhythm detector 12 to the information collector unit 14, provided that the detector can be attached to a portion capable of detecting the biological rhythm involved in the voluntary movement preferably without making the subject feel uneasy. However, when measurement is made on a voluntary movement involving the spatial displacement of the whole body over a long distance such as gait, at least the biological rhythm detector 12 and the information collector unit 14 may be integrated with each other to be attached to articles worn regularly by the subject, which is preferable from the viewpoint of portability. For example, the regularly used articles include eyeglasses, caps, clothes, shoes, belts, watches, bags, accessories, or portable telephones, which the integrated biological rhythm detector 12 and information collector unit 14 are attached to, connected to, or stored in.

Now, data analyses according to the fractal analysis, FFT analysis, autocorrelation function analysis, nonlinear analysis (chaotic attractor), Lyapunov exponent, and scaling analysis based on principal component analysis will be described in detail in this order.

1) Fractal Analysis

It is believed that the biological rhythm produced by voluntary movement is controlled by the CPG (Central Pattern Generator) which is present in the neurological circuit including the spinal cord. However, the biological rhythm is not provided with a constant frequency and affected by a variation in condition of or in environment surrounding the subject to thereby vary delicately. The fractal analysis is one of the techniques for quantitatively analyzing the variation.

First, the frequency of the biological rhythm is extracted from a change in acceleration of the biological rhythm sampled at a sampling frequency which is high enough to reproduce the original data. If the change in acceleration is a repetitive waveform with peaks, the time intervals of the peaks can be calculated and thus a change in frequency of the biological rhythm can be extracted.

Then, the fractal analysis is carried out on the time-series frequencies of the biological rhythm. In general, biological signals contain much noise and are non-stationary signals having statistics values, such as mean or variance, which vary with time, and therefore noise and trend are eliminated from the biological signals. Thereafter, a window is set which is variable with respect to the time axis, and the magnitude of a fluctuation of the signal within the window is calculated. More specifically, noise is eliminated according to the equation (1) shown below, and then a fluctuation is calculated after the trend has been eliminated according to the equation (2).

$$\begin{matrix}g ( k ) = {\sum }_{i = 1}^{k} ( X ( i ) - Xavg ) & ( 1 ) \\ S ( n ) = \sqrt{\frac{1}{N} {\sum }_{k = 1} ( g ( k ) - {{g}_{n} ( k )}^{2} )} & ( 2 )\end{matrix}$$

In the equations (1) and (2), x(i) is the time-series data of the frequencies (intervals) of the biological rhythm, Xavg is the mean value, g(k) is an accumulated value of the time-series frequencies with an average of zero, gₙ(k) is a straight line trend with the window width of the time axis being n, and S(n) is the magnitude of the fluctuation with the trend having been eliminated.

In the log-log plot of the resulting fluctuation component S(n) versus the window size n, it is known that the following straight lines are provided: a straight line with a gradient of 0-5 to 1.0 for the time-series data with a long-range correlation; a straight line with gradient of 0.5 for white noise; and a straight line of a gradient of 1-5 for brown noise. It has been reported that a variation in heartbeat of a person shows a fluctuation referred to as a so-called “1/f fluctuation” . For this 1/f fluctuation, the log-log plot of S(n) versus the window side n provides a straight line of a gradient of 1.0. In addition, it can be said that the absolute value of S(n) is an index representing the performance of biological control since the absolute value shows a variation in frequency of biological rhythms with the trend having been eliminated.

2) FFT Analysis

The FFT analysis is an analytical technique for showing quantitatively how much power is provided for a triangular function (sinusoidal wave) of a certain frequency existing in given arbitrary time-series signals. This is based on the theory of Fourier series that a given periodic function can be expressed with the sum of triangular functions having different frequencies. Specifically, frequency spectrum X(f) can be obtained through an operation on the time-series data x(t) in accordance with the equation (3).

$$S ( n ) = {\int }_{- ∞}^{∞} X ( t ) \cdot {ⅇ}^{i2πft} ⅆ t \qquad ( 3 )$$

3) Autocorrelation Function

The autocorrelation function shows quantitatively how much the waveform x(t+τ) shifted by the time “τ” with respect to the time-series data x(t) resembles to the original waveform. The autocorrelation function is employed for estimating a frequency component in the time-series data or for determining how far in time the correlation can be held. The autocorrelation function R(τ) is obtained through an operation on the time-series data x(t) in accordance with the equation (4).

R(τ)=1/NΣX(t)·X(t+τ) (4)

4) Nonlinear Analysis (chaotic attractor)

It is conceived that a multi-variable control system is configured to produce biological rhythms. However, it is impossible to measure the status of all variables. Nevertheless, on the presumption that these multiple variables function under a certain relationship, it is made possible to estimate the status of other variables from the status of a limited number of variables. This is the relationship known as the Takens' embedding theorem. This theorem allows the status space of multiple variables to be reproduced based on the measurements of a limited number of variables in accordance with sampling by employing delay time. The trajectory of the status space is called the chaotic attractor, allowing a difference in structure of the multi-variable control system to be recognized as a difference in trajectory of the chaotic attractor.

5) Lyapunov Exponent

It is known that a time series moving apparently at random has three features if the generation mechanism follows certain determinism. The three features include:

(a) Divergence of trajectories (attractor sensitiveness to initial value);

(b) (Medium- and long-range) unpredictability; and

(c) Self-similarity.

Among those features, the Lyapunov exponent expresses quantitatively the “divergence of trajectories” and the “(medium- and long-range) unpredictability”. The Lyapunov exponent indicates how far two adjacent attractors are separated from each other after a certain time. The relationship shown in the equation (5) holds, where y1(t) is one trajectory of the attractor, y2(t) is another trajectory, and λ is Lyapunov exponent. For positive Lyapunov exponents λ, the attractor trajectory becomes unstable, and the time-series is shown to be possibly chaotic.

|y1(t+Δt)−y2(t+Δt)|=e⁸⁰|y1(t)−y2(t)| (5)

6) Scaling Analysis Based on Principal Component Analysis

The principal component analysis is one of the multivariate analyses, being applied to extraction of a trend from multi-dimensional data

First, the frequency of the biological rhythm is extracted from a change in acceleration of the biological rhythm sampled at a sampling frequency high enough to reproduce the original data. If the change in acceleration is a repetitive waveform with peaks, the time intervals of the peaks can be calculated and thus a change in frequency of the biological rhythm can be extracted. Thus, the time series frequencies of the biological rhythm are determined Then, the time-series data is divided based on and to have a variable window width h, thereby providing n sets of data in total. A matrix X with h columns and n rows each having a set of data is created, and then covariance matrix cov(X) is calculated from the matrix X in accordance with the equation (6).

cov(X)p=X^{T}X/(n−1) (6)

Then, the maximum eigenvalue λ and the eigenvector p of the covariance matrix are determined to satisfy the relationship of the equation (7).

cov(X)p=λp (7)

The resulting maximum eigenvalue λ corresponds to the variance of the principal component of the time-series data and is an index representing the fluctuation of data. Thus, it is conceivable that the log-log plot of λ versus the window width h shows the scale dependency of biological rhythm data and reflects the performance of biological control.

Now the present invention will be described with reference to examples.

### FIRST EXAMPLE

In this example, a gait rhythm was measured. First, as shown in FIG. 3, a portable device was prepared which was to detect and collect data on a biological rhythm.

The gait rhythm was detected with a small strain-gauge acceleration sensor 30 (for measuring along three axes, i.e., vertically, sideways, and back and forth with the range of ±5G). The acceleration sensor 30 was attached to the middle dorsolumber portion of a subject without making the subject feel uneasy in order to measure the acceleration at the dorsolumber portion upon gait. The gait place may have some undulations or steps so long as they are not heavily contoured like staircases. For example, use can be made of a park, the subject's home, or roads around the workplace of the subject. In the shoes that the subject usually wears, the subject walks for about 10 minutes at a speed normal to the subject, during which the gait rhythm is measured.

The acceleration sensor 30 may be connected to a data logger 32 directly or with a cable. The data logger 32 is provided with eight input terminals and can collect eight different types of data at the same time; however, in this embodiment, only three channels are used corresponding to the output from the three-axis acceleration sensor 30. During the gait, the subject carried the data logger 32 attached to the belt or the like, thereby allowing the gait rhythm to be collected in real time. The sampling frequency may be preferably varied within the range from 0.2 Hz to 1000 Hz. In this example, sampling was carried out at 100 Hz. The data was recorded on a compact flash memory card (192 MB in capacity) which was provided in a data collector unit 32.

Data Analysis

(1) After the gait, the compact flash memory card was removed from the data collector unit 32 and then connected to a computer for data analysis.

At the instant when the heel of a foot touches the around, the peak of a reactive force exerted on the body from the ground appears in the vertical acceleration waveform measured on the middle dorsolumber portion during the gait. Thus, the peak intervals provide the time-series data on gait intervals. Alternatively, since sudden changes in back-and-forth acceleration waveform appear corresponding to the position of the peaks, the positions where the most sudden change appears may be extracted to determine the intervals as gait intervals.

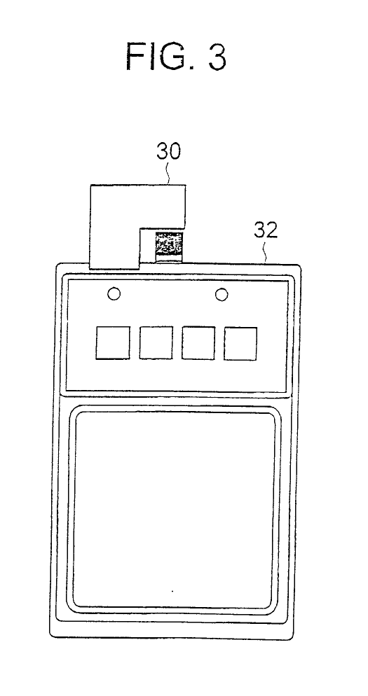

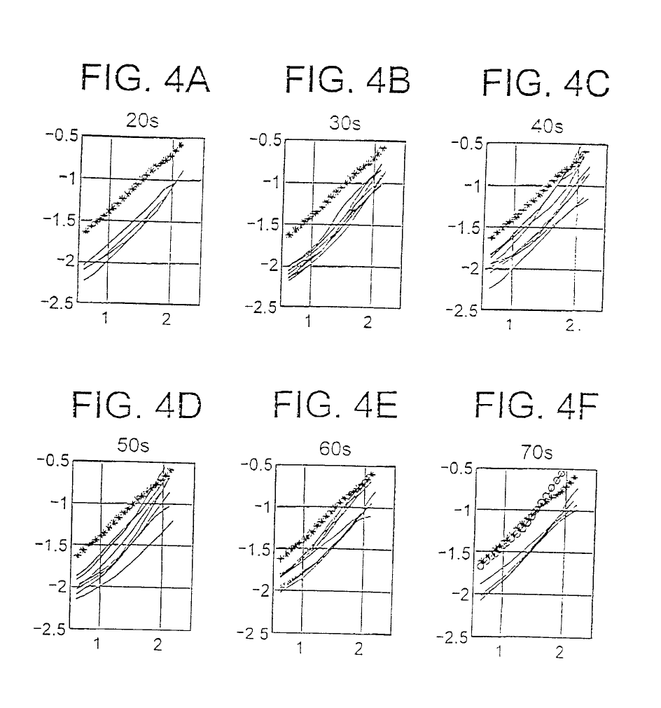

Fractal analysis was carried out on the time-series data of the gait intervals and then the resulting data is shown in FIGS. 4(a) to (f) as log-log plots of fluctuation component S(n) versus window size n. FIGS. 4(a) to (f) show the data of subjects in their twenties, thirties, forties, fifties, sixties, and seventies. In these figures, the horizontal axis represents log₁₀(n) and the vertical axis represents log₁₀S(n).

The subjects were fourty-two able-bodied persons and two patients with a reduced neurological physiological function, FIG. 4 showing the graphs each representing data for each group of the subjects in their twenties to seventies. As shown in the figures, the patients with a reduced neurological physiological function are shown with marks “*” and “o”. Mark “*” represents a female patient in her sixties whom her doctor has diagnosed as having Parkinson's disease and is plotted in all graphs for comparison. Mark “o” represents a male person who has been measured as an able-bodied person at first but diagnosed after this experiment as having an earlier stage dementia disease through a detailed inspection conducted by his doctor.

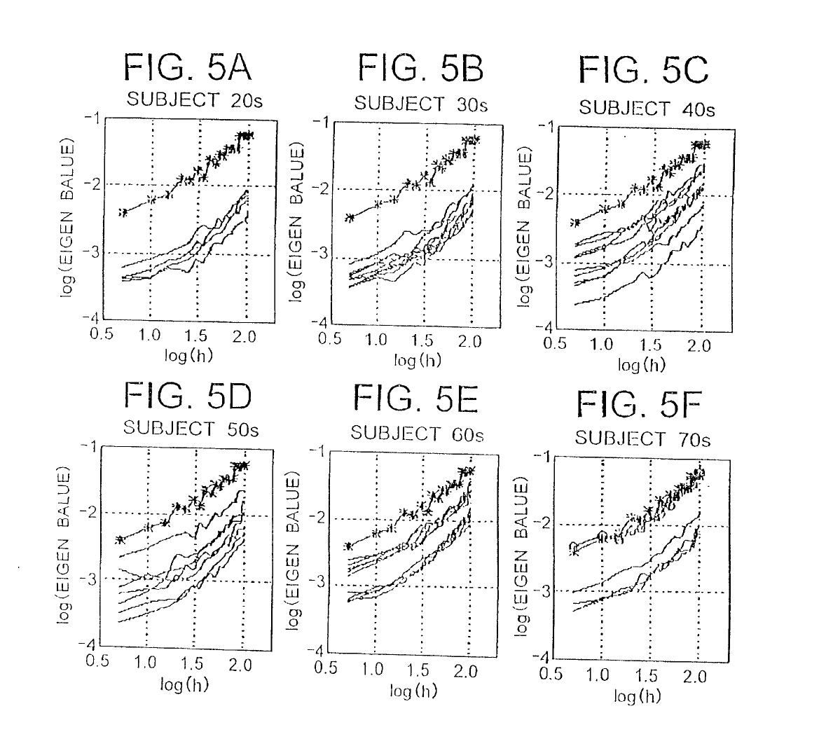

As can be seen from the graphs, the absolute values obtained through the fractal analysis indicate a distinct difference between the able-bodied and the patients with a reduced neurological physiological function over all scales (all window sizes n) It can also be found from the tendency of the fractal analysis that some able-bodied persons in their forties to sixties are gradually coming close to the patients with a reduced neurological physiological function. The absolute value of the fractal analysis S(n) represents a variation in biological rhythm frequency with the trend having been eliminated and therefore can be taken as an index representing aging and the performance of biological control (Index for able-bodied <Index for patients with reduced neurological physiological function) A scaling analysis based on principal component analysis has been conducted on the same data about gait intervals and then the resulting data is shown in FIG. 5 as log-log plots of the maximum eigenvalue (λ) versus the window width h. Like the results of the fractal analysis, as can be seen from the graphs, the absolute values obtained through the scaling analysis indicate a distinct difference between the able-bodied and the patients with a reduced neurological physiological function. Moreover, when compared with the fractal analysis, a clear separation can be found between the able-bodied and the patients with a reduced neurological physiological function over all scales (window sizes).

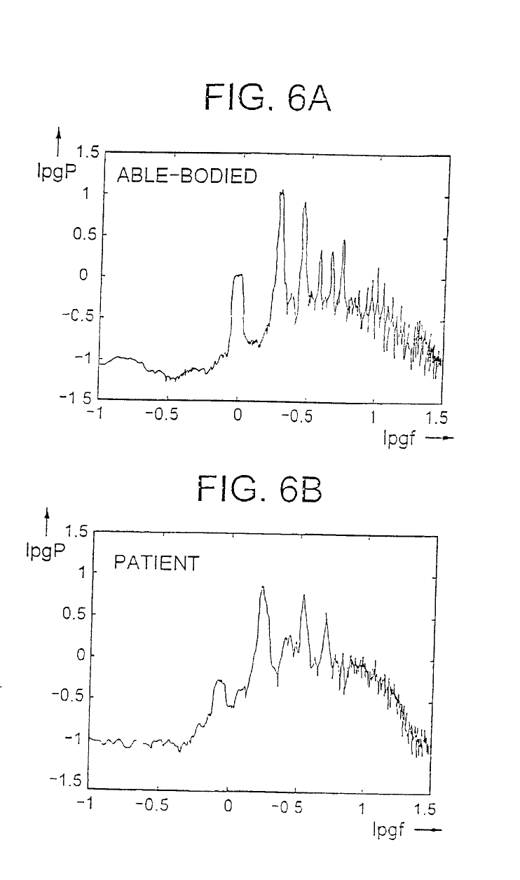

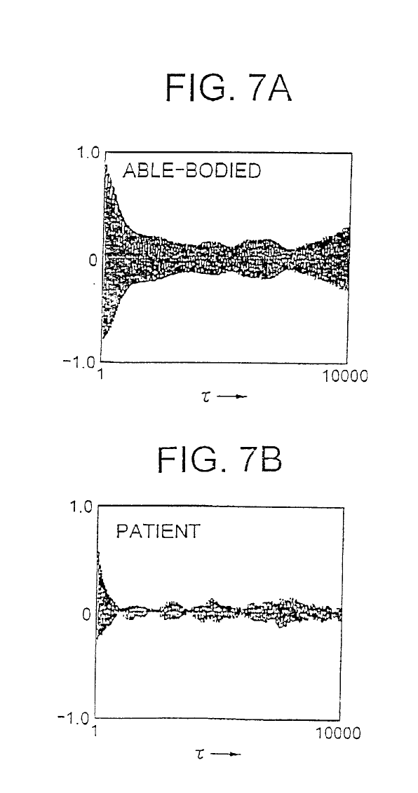

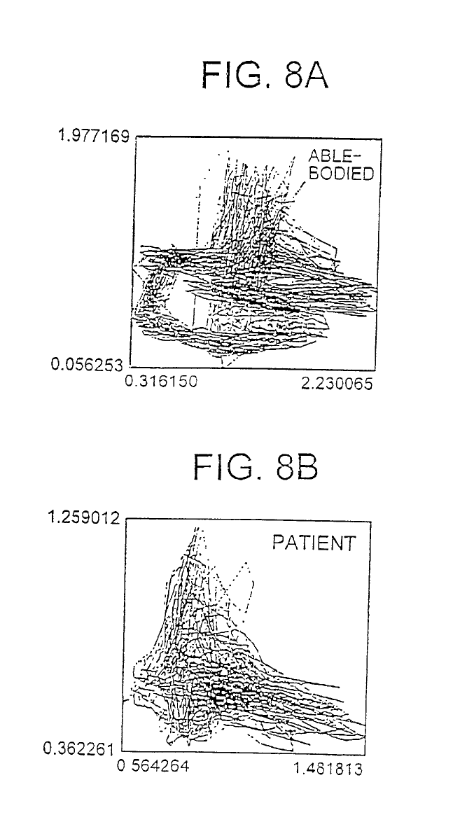

(2) FIGS. 6(a) and (b) show the results obtained through a FFT analysis conducted on the raw vertical acceleration data measured on the middle dorsolumber portion. FIG. 6(a) shows the results of the analysis of the able-bodied, whereas FIG. 6(b) shows those suffered from Parkinson's disease. In FIGS. 6(a) and 6(b), the horizontal axis represents log f and the vertical axis represents log P. Since the human's gait intervals (right step, left step, right step, and so on) are about 0.5 seconds, the maximum power is indicated at 2 Hz. As can be seen from FIGS. 6(a) and (b), multiple peaks appear on the able-bodied, whereas a peak appears at about 2 Hz for the patients with a reduced neurological physiological function, with no harmonic components being found except for two powers appearing at 4 to 6 Hz.

(3) FIGS. 7(a) and (b) each shows an autocorrelation function of the acceleration data on the biological rhythm of the able-bodied persons and patients with a reduced neurological physiological function. The vertical axis represents the correlation factor and the horizontal axis represents a shift in time upon calculation of the correlation factor (τ in the equation (4)). The able-bodied indicates a long-term correlation, whereas the patients with a reduced neurological physiological function do not.

(4) FIGS. 8(a) and (b) each shows a chaotic attractor of the acceleration data on the biological rhythm of the able-bodied and the patients with a reduced neurological physiological function. The chaotic attractor has been drawn using software (“CHORUS” by Computer Convenience), which is commercially available for the chaotic analysis. Settings have been made to an embedded delay time of 50 milliseconds, to four-dimensional embedded dimensions, and to an eye-point 178 degrees in vertical axis and 85 degrees in horizontal axis. The attractor of the able-bodied indicates trajectories with a space formed in the middle thereof, whereas the trajectories of the patients with a reduced neurological physiological function concentrate in a spot indicating no distinct trajectories.

(5) The first Lyapunov exponent of the chaotic attractor having the aforementioned embedded delay time of 50 milliseconds and embedded in the four dimensions is 0.033 for the able-bodied and 0.212 for the patients with a reduced neurological physiological function. Like the fractal analysis, this makes it possible to take the chaotic attractor as an index for representing the performance of biological control.

As described above, there have been developed a diagnostic method and apparatus for people with a reduced neurological physiological function. The method and apparatus make it possible to diagnose the probability of suffering from a disease objectively by analyzing the data obtained non-invasively from the able-bodied and the patients with a reduced neurological physiological function and by comparing the data with those of the able-bodied. This also allows those other than a specialist doctor to diagnose the disease of a patient with a reduced neurological physiological function with a high probability at an earlier stage, in a non-invasive manner, and objectively

### SECOND EXAMPLE

This is another example for the embodiment in which the gait rhythm is measured as a target rhythm. In the manner similar to that in the first embodiment, the gait rhythm was measured and the fractal analysis was carried out on the resulting data. However, in this example, time-series variations in gait rhythm of the same subject were traced, and the correlation between the resulting data and the physical condition of the subject was discussed.

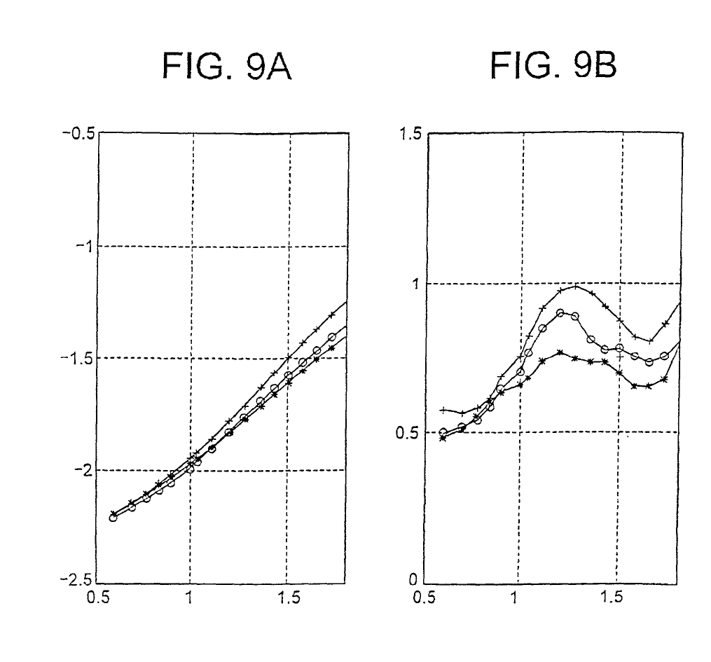

FIG. 9(a) shows a log-log plot of the fluctuation component S(n) of the gait rhythm versus window size n, and FIG. 9(b) shows its differentiated plot. As illustrated, mark “+” designates the results obtained under a good condition of the subject, whereas mark “*” designates the measurements conducted three days later, when the subject had a cold and not in a serious condition however. In addition, mark “o” designates the measurements conducted on the following day when the subject is getting better. From these graphs, any significant difference can be hardly found in the absolute value of the fluctuation of the fractal analysis. However, a distinct difference can be found to exist in differential value near 1 to 1.5 on the horizontal axis (Differential during the cold<Differential under good physical condition or recuperation from the cold).

This makes it possible to diagnose a reduction and recuperation in physiological function at an earlier stage with high probability, in a non-invasive manner, and objectively.

The present embodiment implements a biological rhythm inspection method for determining a variation or aging in physiological function at an earlier stage. The method includes the steps of recording and storing biological rhythm information obtained by measuring as biological rhythm information the repetitive rhythmic muscle activity caused by a voluntary movement or the muscle activity involved in the repetitive rhythmic movement. The method also includes the step of comparing the resulting data obtained through analysis on the biological rhythm information with the past data of the subject or other person. The present invention also realizes an inspection apparatus for allowing the biological rhythm inspection method to be readily implemented. The biological rhythm inspection apparatus includes a biological rhythm detector unit for non-invasively measuring the muscle activity involved in a repetitive rhythmic movement caused by a person's voluntary movement, and an information collector unit for recording and storing the resulting biological rhythm information obtained from the biological rhythm detector unit.

Since the above embodiments are described only for examples, the present invention is not limited to the above embodiments and various modifications or alterations can be easily made therefrom by those skilled in the art without departing from the scope of the present invention.

What is claimed is:

1. A biological rhythm inspection apparatus comprising:
a biological rhythm detector for non-invasively measuring a muscle activity in a repetitive rhythmic movement caused by a voluntary movement involving a body movement of a subject;
an information collector unit for recording or storing biological rhythm information obtained from the biological rhythm detector; and
an analyzing unit for analyzing a variation or aging in physiological function based on the biological rhythm information.

2. The biological rhythm inspection apparatus according to claim 1, wherein the analyzing unit is an information processing unit.

3. A biological rhythm inspection method comprising the steps of:
measuring a repetitive rhythmic muscle activity caused by a voluntary movement involving a body movement of a subject, by using a biological non-invasive rhythm detector attached on a body of the subject, to obtain biological rhythm information;
comparing the biological rhythm information with reference data; and
analyzing a variation or aging in physiological function of the subject based on the result of the comparison.

4. The biological rhythm inspection method according to claim 3, wherein an acceleration sensor is used as the biological non-invasive rhythm detector.

5. The biological rhythm inspection method according to claim 3, wherein the analyzing step is conducted based on a fractal analysis, an FFT analysis, an autocorrelation function analysis, a nonlinear analysis, a Lyapunov exponent, and/or a scaling analysis.

6. The biological rhythm inspection method according to claim 3, wherein the voluntary movement is a gait movement.

7. The biological rhythm inspection method according to claim 3, wherein the variation or aging in physiological function is a neurological disease.
